(19) United States
(12) Patent Application Publication
(10) Pub. No.: US 2015/0328222 A1
(43) Pub. Date: Nov. 19, 2015

(54) PAIN RELIEF CREAM

(71) Applicants: Paul J. Tortoriello, Dyer, IN (US); Kristin M. Tortoriello, Dyer, IN (US)

(72) Inventors: Paul J. Tortoriello, Dyer, IN (US); Kristin M. Tortoriello, Dyer, IN (US)

(21) Appl. No.: 14/715,151

(22) Filed: May 18, 2015

Related U.S. Application Data
(60) Provisional application No. 61/996,917, filed on May 19, 2014.

(51) Int. Cl. A61K 31/5375; A61K 47/22; A61K 47/14; A61K 47/44; A61K 9/00; A61K 47/46

(52) U.S. Cl. CPC A61K 31/5375; A61K 9/0014; A61K 47/46; A61K 47/14; A61K 47/44; A61K 47/22

(57) ABSTRACT

Compositions for topically treating painful skin conditions are described. The compositions comprise a water phase component comprising a pharmaceutically effective amount of a pramoxine compound, a moisturizer and water; an oil phase component comprising a natural butter, a carrier oil and an emollient, and an emulsifier.

### CROSS-REFERENCE TO RELATED APPLICATIONS

This application claims priority to U.S. Provisional Application No. 61/996,917, filed May 19, 2014, the entire contents of which are hereby incorporated by reference in their entirety.

### TECHNICAL FIELD

The present technology relates generally to pain relief compositions, systems and methods. In particular, the present technology relates to pain relief compositions containing an analgesic.

### BACKGROUND

The following description is provided to assist the understanding of the reader. None of the information provided or references cited is admitted to be prior art.

Topical anesthetics or analgesics are used to relieve pain and itching caused by conditions such as hemorrhoids, minor burns, insect bites, cuts and scratches. Many topical analgesic agents such as lidocaine, prilocaine, xylocaine, benzocaine and the like are used to treat pain relief However, while many of the available formulations containing these agents are effective in treating pain and discomfort by suppressing sensitization, they fail to promote healing or protect the skin from further breakdown. New pain relief compositions must, therefore, find a balance between greater pain treatment efficacy and effective healing and protection. Most of the presently available commercial products fail to optimize either attribute.

### SUMMARY

In accordance with one aspect, the present technology relates to compositions for treating skin conditions including painful skin conditions. In one embodiment, the composition includes a water phase component which includes a pharmaceutically effective amount of a pramoxine compound, a moisturizer and water; an oil phase component which includes a natural butter, a carrier oil and an emollient, and an emulsifier.

In some embodiments, the pramoxine compound is pramoxine hydrochloride which is present in an amount of from about 0.25 to about 1.5 wt. % relative to the total volume of the composition.

In some embodiments, the composition includes pramoxine hydrochloride, shea butter, aloe vera, lanolin, grape seed oil, emulsifying wax and water. In some embodiments, the composition further includes one or more components selected from a group consisting of antioxidants, preservatives, and essential oils. In some embodiments, the antioxidant is Vitamin E. In some embodiments, the preservatives are grapefruit seed extract and sodium borate. In some embodiments, the essential oils are lavender oil and chamomile oil.

In some embodiments, the composition includes (a) about 0.25% to about 1.5% by weight of pramoxine hydrochloride, (b) about 10% to about 40% by volume of grape seed oil, (c) about 10% to about 25% by volume of aloe vera, (d) about 5% to about 20% by volume of lanolin, (e) about 5% to about 10% by volume of shea butter, (f) about 0.1% to about 10% by volume of emulsifying wax, (g) about 0.1% to about 5% by volume of vitamin E, and (h) water to make 100%.

In one aspect the present technology provides a method for topically treating painful skin conditions, wherein the method comprises applying a topical composition to skin wherein the composition includes a water phase component which includes a pharmaceutically effective amount of a pramoxine compound, a moisturizer and water; an oil phase component which includes a natural butter, a carrier oil and an emollient; and an emulsifier.

In another aspect the present technology provides a method for preparing a topical composition, which includes heating a water phase component comprising a pharmaceutically effective amount of a pramoxine compound, a moisturizer and water; heating an oil phase component comprising a natural butter, a carrier oil, an emollient and an emulsifier; combining the water phase component and the oil phase component to form an emulsion; and cooling the emulsion.

The foregoing summary is illustrative only and is not intended to be in any way limiting. In addition to the illustrative aspects, embodiments and features described above, further aspects, embodiments and features will become apparent by reference to the following drawings and the detailed description.

### DETAILED DESCRIPTION

In the following detailed description, the illustrative embodiments described in the detailed description, figures, and claims are not meant to be limiting. Other embodiments may be utilized, and other changes may be made, without departing from the spirit or scope of the subject matter presented here. In the description that follows, a number of terms are used extensively. The terms described below are more fully understood by reference to the specification as a whole. Units, prefixes, and symbols may be denoted in their accepted SI form.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

The expression “comprising” means “including, but not limited to.” Thus, other non-mentioned substances, additives, carriers, or steps may be present. Unless otherwise specified, “a” or “an” means one or more.

As used herein, the term “and/or” includes any and all combinations of one or more of the associated listed items.

The term “about” when used before a numerical designation, e.g., temperature, time, amount, and concentration, including range, indicates approximations which may vary by (+) or (−) 10%, 5% or 1%.

As used herein, the term “contacting” encompasses the placement of the composition, directly or indirectly, on to the skin surface.

As used herein, the term “combining” refers to the mixing or admixing of ingredients in a composition or formulation.

The present technology provides compositions and products which include a topical analgesic and methods of using these compositions to treat skin conditions including painful skin conditions and other skin disorders. In one aspect, provided is a topical composition which includes a water phase component, an oil phase component and an emulsifier. The water phase component may include a topical analgesic or anesthetic. Suitable analgesic or anesthetic compounds are those which provide a pain relieving effect. Exemplary topical analgesic or anesthetic compounds include, but are not limited to, pramoxine, lidocaine, benzocaine, prilocaine, lignocaine, xylocaine, bupivacaine, tetracaine, dyclonine, dibucaine, xylocaine, ketocaine, clove oil, methyl salicylate and trolamine salicylate, and the like or salts thereof, or combinations of two or more thereof In one embodiment, the topical analgesic is a pramoxine compound. In one embodiment, the topical analgesic is a water-soluble salt of pramoxine. In one embodiment, the topical analgesic is a pharmaceutically acceptable salt of pramoxine. In one embodiment, the topical analgesic is pramoxine hydrochloride (pramoxine HCl).

Pramoxine is the common name of 4-[3-(4-Butyoxyphenoxy) propyl] morpholine. Pramoxine is commonly used in the form of pramoxine HCl which is water soluble and has a low toxicity profile. Pramoxine HCl provides beneficial topical analgesic or anesthetic effects with a low incidence of sensitivity or reactions. A percentage of 0.5-1% w/v pramoxine HCl has been approved for topical application by the FDA. It is believed that the mechanism by which pramoxine works is by temporarily blocking both the initiation, as well as the conduction of sensory nerve impulses, leading to numbness.

Suitable pramoxine compound, such as pramoxine HCl may be present in an amount from about 0.001 to about 5 wt. % relative to the total volume of the composition. This includes from about 0.01 wt. % to about 4 wt. %, about 0.1 wt. % to about 3 wt. %, about 0.3 wt. % to about 2 wt. %, about 0.5 wt. % to about 1.5 wt. %, about 0.8 wt. % to about 1.2 wt. %, about 0.9 wt. % to about 1.1 wt. %, and ranges between any two of these values or less than any one of these values. In some embodiments, the pramoxine hydrochloride is present in an amount of from about 0.25 to about 2 wt. % relative to the total volume of the composition. In some embodiments, the pramoxine hydrochloride is present in an amount of from about 0.5 to about 1.5 wt. % relative to the total volume of the composition. In some embodiments, a pramoxine compound, such as pramoxine hydrochloride, is present in an amount of about 1 wt. %.

In addition to the pramoxine compounds, the water phase component may include a moisturizer. Suitable moisturizers include, but are not limited to, aloe vera, glycerin, water soluble polyols, propylene glycol, polyethylene glycol, polypropylene glycol, sorbitol, and pantothenol, and the like or a combination of two or more thereof Suitable polyols include glycerin, sorbitol, mannitol, maltitol, isomalt, xylitol, and erythritol. In some embodiments the moisturizer is aloe vera. Other moisturizers include mineral oil. In some embodiments, the aloe vera is aloe barbadensis leaf extract (e.g., pure aloe barbadensis leaf extract). The aloe vera can be in the form of a gel or liquid or any other suitable form. In some embodiments, the aloe vera is present as aloe vera liquid. In addition to moisturizing, the polysaccharides such as glucomannan and acemannan present in aloe vera may impart other beneficial properties to the composition such as antibiotic, anti-inflammatory, wound healing, soothing and calming of skin.

The water phase component of the present compositions may include a suitable moisturizer, such as aloe vera in an amount from about 0.1 to about 50 vol. % relative to the total volume of the composition. This includes from about 1 vol. % to about 40 vol. %, about 5 vol. % to about 30 vol. %, about 10 vol. % to about 25 vol. %, about 15 vol. % to about 20 vol. %, about 17 vol. % to about 19 vol. %, and ranges between any two of these values or less than any one of these values. In some embodiments, the aloe vera is present in an amount of from about 10 to about 25 vol. % relative to the total volume of the composition. In some embodiments, the aloe vera is present in an amount of from about 15 to about 20 vol. % relative to the total volume of the composition. In some embodiments, a moisturizer, such as aloe vera, is present in an amount of about 15 vol. %, about 16 vol. %, about 17 vol. %, about 18 vol. %, about 19 vol. % or about 20 vol. %. In some embodiments, a moisturizer, such as aloe vera, is present in an amount of about 17.9 vol. %.

The compositions described in the present technology further include water. In some embodiments, the water may be purified water. In some embodiments, the compositions further comprise sufficient water to make 100% by weight or volume. In some embodiments, the composition comprises about 5% to about 60%, about 10% to about 50%, about 15% to about 40%, about 20% to about 35%, about 25% to about 30% by volume water. In some embodiments, the composition comprises about 20% to about 30% by volume water. In some embodiments, water is present in an amount of about 25 vol. %, about 26 vol. %, about 27 vol. %, about 28 vol. %, about 29 vol. %, about 30 vol. %, about 31 vol. % or about 32 vol. %. In some embodiments, water is present in an amount of about 29.8 vol. %.

The oil phase component of the present compositions may include a suitable natural butter. Natural butters used in the present compositions may include, for example, fats derived from various natural sources such as plant parts including seeds, nuts and fruits. Suitable natural butter includes, but is not limited toselected from the group consisting of shea butter, cocoa butter, aloe butter, olive butter, avocado butter, sweet almond butter, shealoe butter, coffee bean butter, cupuacu butter, coconut butter, hemp seed butter, kokum butter, macadamia nut butter, illipe butter, mango butter, mochacchino butter, murumuru butter, and pistachio nut butter, or a combination of two or more thereof. In some embodiments, the natural butter is shea butter. In some embodiments, the natural butter is hydro-dispersible shea butter.

Shea Butter is a natural butter or naturally occurring fat extracted from the nuts of the Shea fruits using a suitable extraction solvent. Shea butter imparts beneficial properties as a moisturizer and emollient to protect skin from dehydration and other climatic influences, improves appearance of dry, irritated skin by restoring suppleness and smoothness and may act as an anti-inflammatory agent.

The oil phase component of the present compositions may include a suitable natural butter, such as hydro-dispersible shea butter in an amount from about 0.1 to about 30 vol. % relative to the total volume of the composition. This includes from about 1 vol. % to about 25 vol. %, about 2 vol. % to about 20 vol. %, about 3 vol. % to about 15 vol. %, about 5 vol. % to about 10 vol. %, about 7 vol. % to about 9 vol. %, and ranges between any two of these values or less than any one of these values. In some embodiments, the shea butter is present in an amount of from about 1 to about 20 vol. % relative to the total volume of the composition. In some embodiments, the shea butter is present in an amount of from about 5 to about 10 vol. % relative to the total volume of the composition. In some embodiments, a natural butter, such as shea butter, is present in an amount of about 5 vol. %, about 6 vol. %, about 7 vol. %, about 8 vol. %, about 9 vol. % or about 10 vol. %. In some embodiments, a natural butter, such as shea butter, is present in an amount of about 8 vol. %.

In some embodiments, the oil phase component of the present compositions includes an emollient. Suitable emollients include, but are not limited to lanolin, glycerol, lanolin derivatives mineral oil, petrolatum, cholesterol, isostearyl neopentanoate, octyl stearate, mineral oil, isocetyl stearate, myristyl myristate, octyl dodecanol, dimethicone, phenyl trimethicone, cyclomethicone, C₁₂-C₁₅ alkyl benzoates, dimethiconol, propylene glycol, lactic acid, butylene glycol, sodium PCA, carbowax, castor oil, squalane, silicons such as dimethicone, cyclomethicone, simethicone, and urea or a combination of two or more thereof. Suitable lanolin derivatives include, for example, lanolin oil, lanolin wax, lanolin alcohols, lanolin fatty acids, isopropyl lanolate, ethoxylated lanolin, ethoxylated lanolin alcohols, ethoxolated cholesterol, propoxylated lanolin alcohols, acetylated lanolin, acetylated lanolin alcohols, lanolin alcohols linoleate, lanolin alcohols recinoleate, acetate of lanolin alcohols recinoleate, acetate of lanolin alcohols recinoleate, acetate of ethoxylated alcohols esters, hydrogenolysates of lanolin, hydrogenated lanolin, ethoxylated hydrogenated lanolin, ethoxylated sorbitol lanolin, and liquid and semisolid lanolin, In some embodiments, the emollient is selected from the group consisting of lanolin, lanolin derivatives mineral oil, petrolatum, castor oil, squalane, and silicons or a combination of two or more thereof In some embodiments, the emollient is lanolin. The lanolin may be purified lanolin.

Lanolin is a wax secreted by the sebaceous glands of wool-bearing animals. Lanolin is an emollient with moisturizing properties. It may also act as an emulsifier since it has high water absorption capability. Lanolin may soothe and protect dry cracked skin, provide effective moisture barrier, and offer immediate relief from discomfort.

In some embodiments, the oil phase component of the present compositions may include a suitable emollient, such as purified lanolin in an amount from about 0.1 to about 60 vol. % relative to the total volume of the composition. This includes from about 0.5 vol. % to about 50 vol. %, 1 vol. % to about 40 vol. %, 1.5 vol. % to about 35 vol. %, about 2 vol. % to about 30 vol. %, about 5 vol. % to about 20 vol. %, about 10 vol. % to about 15 vol. %, about 12 vol. % to about 14 vol. %, and ranges between any two of these values or less than any one of these values. In some embodiments, the lanolin is present in an amount of from about 12.5 to about 50 vol. % of the total composition. In some embodiments, the lanolin is present in an amount of from about 5 to about 20 vol. % relative to the total volume of the composition. In some embodiments, the lanolin is present in an amount of from about 10 to about 15 vol. % relative to the total volume of the composition. In some embodiments, the lanolin is present in an amount of from about 12 to about 20 vol. % relative to the total volume of the composition. In some embodiments, an emollient, such as lanolin, is present in an amount of about 10 vol. %, about 11 vol. %, about 12 vol. %, about 13 vol. %, about 14 vol. % or about 15 vol. %. In some embodiments, an emollient, such as lanolin, is present in an amount of about 12.8 vol. %.

In some embodiments, the oil phase component of the present compositions includes a carrier oil or a base oil. Suitable carrier oils include, but are not limited to grape seed oil, jojoba oil, sweet almond oil, olive oil, sunflower oil, rosehip seed oil, flax seed oil, safflower oil, argan oil, coconut oil, and avocado oil, or a combination of two or more thereof. In some embodiments, the carrier oil is grape seed oil. In some embodiments, the carrier oil is jojoba oil. In some embodiments, the carrier oil is a mixture of grape seed oil and jojoba oil.

Grape seed oil is a vegetable oil pressed from the seeds of various varieties of grapes. Because of its high linoleic acid content, grape seed oil may impart nourishing and moisturizing properties to the composition and may aid in improving and preserving the elasticity of skin by stabilizing collagen and elastin. It also has bioflavonoids called proanthocyanidins as one its components which may impart anti-oxidant properties. Because it is light and thin, grape seed oil can be used as a base or carrier oil for certain compositions in the cream, lotion, deodorant or other such forms.

Jojoba oil can be obtained by extraction of jojoba (Simmondsia chinensis) seeds with an organic solvent. Because of its similarity with natural skin and body oils, jojoba oil can be used as a base or carrier oil for certain compositions in the cream, lotion, or other such forms.

In some embodiments, the oil phase component of the present compositions may include a suitable carrier oil, such as grape seed oil, jojoba oil or a combination thereof, in an amount from about 0.1 to about 70 vol. % relative to the total volume of the composition. This includes from about 1 vol. % to about 60 vol. %, about 5 vol. % to about 50 vol. %, about 10 vol. % to about 40 vol. %, about 15 vol. % to about 30 vol. %, about 20 vol. % to about 25 vol. %, and ranges between any two of these values or less than any one of these values. In some embodiments, the grape seed oil is present in an amount of from about 10 to about 40 vol. % relative to the total volume of the composition. In some embodiments, the grape seed oil is present in an amount of from about 15 to about 30 vol. % relative to the total volume of the composition. In some embodiments, a carrier oil, such as grape seed oil, is present in an amount of about 20 vol. %, about 21 vol. %, about 22 vol. %, about 23 vol. %, about 24 vol. % or about 25 vol. %. In some embodiments, a carrier oil, such as grape seed oil, is present in an amount of about 23.9 vol. %.

In some embodiments, the compositions described in the present technology further include emulsifiers to increase the stability of the emulsion. Suitable emulsifiers include, but are not limited to, non-ionic and a cationic emulsifiers or surfactants such as emulsifying wax, beeswax, polysorbates, ceteareths, paraffin wax, glyceryl stearate, cetyl alcohol, cetearyl alcohol, cyclodextrins, cetyltrimethylammonium bromide, lecithin, polyoxyl castor oil, butylene glycol, and polyoxyethylene sorbitan, and the like or a combination of two or more thereof. In some embodiments, the emulsifier is emulsifying wax. In some embodiments, the emulsifier is beeswax wax. In some embodiments, the emulsifier includes emulsifying wax and beeswax. The emulsifiers may be included in the oil phase component, water phase component, both phases or added separately. In one embodiment, the emulsifier is included in the oil phase component.

Emulsifying wax includes mixtures of fatty acids of about 12 to 24 carbon atoms in length. Emulsifying waxes that are preferred for use in cosmetic formulations are those that meet the standards of the National Formulary (N. F.). A N. F. grade emulsifying wax may be prepared from cetostearyl alcohol containing a polyoxyethylene derivative of a fatty ester of sorbitan. Beeswax, which is a natural wax produced by honey bees, is also an emulsifier which aids in regulating the consistency of the composition, enhances the elasticity of skin and is noncomedogenic.

In some embodiments, the compositions may include a suitable emulsifier, such as emulsifying wax, in an amount from about 0.01 to about 30 vol. % relative to the total volume of the composition. This includes from about 0.1 vol. % to about 25 vol. %, about 0.5 vol. % to about 20 vol. %, about 1 vol. % to about 15 vol. %, about 2 vol. % to about 10 vol. %, about 3 vol. % to about 5 vol. %, and ranges between any two of these values or less than any one of these values. In some embodiments, the emulsifier is present in an amount of from about 1 to about 10 vol. % relative to the total volume of the composition. In some embodiments, the emulsifier is present in an amount of from about 2 to about 5 vol. % relative to the total volume of the composition. In some embodiments, an emulsifier, such as emulsifying wax, is present in an amount of about 1 vol. %, about 2 vol. %, about 3 vol. %, about 4 vol. %, about 5 vol. % or about 6 vol. %. In some embodiments, an emulsifier, such as emulsifying wax, is present in an amount of about 3.6 vol. %.

In some embodiments, the compositions may include essential oils. Essential oils are oils obtained from plant or animal sources, or their synthetic equivalents. Suitable essential oils include, but are not limited to, lavender oil, chamomile oil, palmarosa oil, jojoba oil, tea tree oil, clary sage oil, coconut oil, cinnamon oil, geranium oil, lemon oil, lime oil, orange oil, sweet orange oil, grapefruit oil, rosemary oil, aniseed oil, eucalyptus oil, camphor oil, calamus oil, cedarwood oil, citronella oil, mint oil, nutmeg oil, vetiver oil, wintergreen oil, ylang-ylang oil, neroli oil, sage oil, sandalwood oil, frankincense oil, ginger oil, peppermint oil, wintergreen oil, jasmine absolute, spearmint oil, patchouli oil, rosewood oil, vanilla oil, lemongrass oil, basil oil, bergamot oil, balsam oils, tangerine oil, Hinoki oil, Hiba oil, ginko oil, eucalyptus oil, pomegranate oil, manuka oil, and calendula oil. Mixtures of one or more essential oils are encompassed by the present technology. In some embodiments, the essential oils may be used to impart fragrance to the composition.

In some embodiments, the one or more essential oils are selected from the group consisting of lavender oil, chamomile oil, tea tree oil, clary sage oil, peppermint oil, spearmint oil and cinnamon oil, and the like or a combination of two or more thereof In some embodiments, the essential oil may include lavender oil, chamomile oil or a combination thereof In some embodiments, the essential oil is French lavender oil. In some embodiments, the essential oil is Roman chamomile oil.

In some embodiments, the essential oils may be added to the compositions in an amount sufficient to impart fragrance characteristics. In various non-limiting embodiments, different concentrations of essential oils from a few drops to a few milliliters may be used. In some embodiments, the concentration of each essential oil in the final products may range from about 0.001% to about 10% (v/v). In some embodiments, the concentration of each essential oil may range from about 0.01% to about 5% (v/v). In some embodiments, the concentration of each essential oil ranges from about 0.1% to about 1% (v/v). In some embodiments, 5-15 drops of each essential oil may be added to the composition. In some embodiments, about 8 drops, about 10 drops, about 11 drops, about 12 drops, about 13 drops, about 14 drops or about 15 drops of each of one or more essential oils is added to the composition. In some embodiments, about 12 drops of each of one or more essential oils is added to the composition.

The compositions of the present technology may further include one or more additive components selected from a group consisting of, pharmaceutically active agents, anti-fungal agents, anti-inflammatory agents, antioxidants, fragrance agents, preservatives, antimicrobial agents, antiseptic agents, coloring agents, thickeners, humectants, stabilizers, pH regulators, conditioners, softeners, skin protectants, skin nutrients, surfactants, essential fatty acids, collagen, elastin, homogenizers, and other suitable additives. In some embodiments, the additives are natural products and do not contain harsh chemicals. These additives, if present as solid, can be incorporated in the compositions at a concentration in the range of about 0.001 wt. %, about 0.01 wt. %, about 0.02 wt. %, about 0.05 wt. %, about 0.1 wt. %, about 0.5 wt. %, about 1 wt. %, about 2 wt. %, about 5 wt. %, about 10 wt. %, about 15 wt. %, about 20 wt. %, and ranges between any two of these values or less than any one of these values. These additives, if present as liquid, can be incorporated in the compositions at a concentration in the range of about 0.001 vol. %, about 0.01 vol. %, about 0.02 vol. %, about 0.05 vol. %, about 0.1 vol. %, about 0.5 vol. %, about 1 vol. %, about 2 vol. %, about 5 vol. %, about 10 vol. %, about 15 vol. %, about 20 vol. %, and ranges between any two of these values or less than any one of these values.

In some embodiments, the preservatives utilized in the present compositions include any suitable preservatives known in the art, including cosmetic grade preservatives. The preservatives may function as antioxidants. Suitable preservatives include, but are not limited to, boric acid or its salts such as sodium borate and potassium borate, sodium acid pyrophosphate, potassium pyrophosphate, sodium benzoate, calcium benzoate, potassium benzoate, potassium sorbate, sodium sorbate, calcium sorbate, sodium acetate, calcium acetate, sodium diacetate, calcium diacetate, sodium propionate, calcium propionate, potassium propionate, niacin, citric acid, sorbic acid, sodium propinate, paraaminobenzoic acid esters (parabens) and the like or mixtures thereof. In some embodiments, the preservative is sodium borate. Boric acid or sodium borate possess good antiseptic properties and are effective preservative against yeast. In some embodiments, the preservative is cosmetic grade sodium borate. For example, the compositions may include an amount of sodium borate in the range of about 0.1% to about 1.5% by weight, commonly about 0.2% to about 1.3% by weight, more typically about 0.5% to about 1% by weight. In some embodiments, the amount of preservative, such as sodium borate in the composition is in the range of about 0.1% to about 2% by weight. In some embodiments, the amount of preservative, such as sodium borate in the composition is in the range of about 0.5% to about 1% by weight. In some embodiments, a preservative, such as sodium borate, is present in an amount of about 0.5 wt. %, about 0.6 wt. %, about 0.7 wt. %, about 0.8 wt. %, about 0.9 wt. % or about 1 wt. %. In some embodiments, a preservative, such as sodium borate, is present in an amount of about 0.75 wt. %.

In some embodiments, the compositions of the present technology include one or more antioxidants. Exemplary antioxidants include, but are not limited to Vitamin E, butylated hydroxyanisol (BHA), butylated hydroxytoluene (BHT), and tertiary butyl hydroquinone (TBHQ), other tocopherols, propyl gallate, ascorbyl palmitate and other cosmetic grade antioxidants, and the like or a combination of two or more thereof. In some embodiments, the antioxidant is Vitamin E. Vitamin E also includes the various derivatives of these compounds. Vitamin E is a fat-soluble antioxidant and is also known to promote healing of skin wounds. The amount of Vitamin E in the composition may range from about 0.001% to about 5% by volume. In some embodiments, the amount of Vitamin E in the composition is in the range of about 0.01% to about 3% by volume. In some embodiments, the amount of an antioxidant, such as Vitamin E, in the composition is in the range of about 0.1% to about 2.5% by volume. In some embodiments, the amount of Vitamin E in the composition is in the range of about 0.5% to about 2% by volume. In some embodiments, an antioxidant, such as Vitamin E, is present in an amount of about 0.5 vol. %, about 1 vol. %, about 1.5 vol. %, about 2 vol. % or about 2.5 vol. %. In some embodiments, an antioxidant, such as Vitamin E, is present in an amount of about 1.5 vol. %.

In some embodiments, the present compositions may also contain additional natural products which perform multiple functions. Thus, for example, the composition may further include grapefruit seed extract which functions as an antimicrobial agent and a natural preservative. Thus, depending on its function, in various embodiments, the amount of grapefruit seed extract in the composition may range from about 0.001% to about 5% by volume. In some embodiments, the amount of grapefruit seed extract in the composition is in the range of about 0.01% to about 2% by volume. In some embodiments, the amount of grapefruit seed extract in the composition is in the range of about 0.1% to about 0.9% by volume. In some embodiments, the amount of grapefruit seed extract in the composition is in the range of about 0.5% to about 0.8% by volume. In some embodiments, a preservative, such as grapefruit seed extract, is present in an amount of about 0.5 vol. %, about 0.6 vol. %, about 0.7 vol. %, about 0.8 vol. %, about 0.9 vol. % or about 1 vol. %. In some embodiments, a preservative, such as grapefruit seed extract, is present in an amount of about 0.75 vol. %.

In some embodiments, the composition includes a water phase component which includes pramoxine hydrochloride, aloe vera, sodium borate and water. In some embodiments, the composition includes an oil phase component which includes lanolin, shea butter, grape seed oil, emulsifying wax and vitamin E. In some embodiments, the composition includes pramoxine hydrochloride, shea butter, aloe vera, lanolin, grape seed oil, emulsifying wax and water.

In one aspect, the present technology provides a topical composition which includes (a) about 0.25% to about 2% by weight of pramoxine hydrochloride, (b) about 10% to about 40% by volume of grape seed oil, (c) about 10% to about 25% by volume of aloe vera, (d) about 5% to about 20% by volume of lanolin, (e) about 1% to about 20% by volume of shea butter, (f) about 0.1% to about 10% by volume of emulsifying wax, (g) about 0.1% to about 5% by volume of vitamin E, and (h) water to make 100% (typically about 15 to about 40% by volume). In some embodiments, the compositions further include one or more of grapefruit seed extract, sodium borate and essential oils. In some embodiments, the compositions include about 0.1% to about 1.5% by volume of grapefruit seed extract. In some embodiments, the compositions include about 0.5 wt. % to about 1 wt. % of a preservative, such as sodium borate. In some embodiments, the compositions include about 5 to 20 drops each of lavender oil and chamomile oil.

In another aspect, the present technology provides a topical composition which includes (a) about 0.5% to about 1.5% by weight of pramoxine hydrochloride, (b) about 15% to about 30% by volume of grape seed oil, (c) about 15% to about 20% by volume of aloe vera, (d) about 10% to about 15% by volume of lanolin, (e) about 5% to about 10% by volume of shea butter, (f) about 2% to about 5% by volume of emulsifying wax, (g) about 0.5% to about 2% by volume of vitamin E, and (h) water to make 100%. In some embodiments, the compositions further include one or more of grapefruit seed extract, sodium borate and essential oils. In some embodiments, the compositions include about 0.5% to about 0.9% by volume of grapefruit seed extract. In some embodiments, the compositions include about 0.6 wt. % to about 0.9 wt. % of a preservative, such as sodium borate. In some embodiments, the compositions include about 10 to 15 drops each of lavender oil and chamomile oil.

In one aspect, the present technology provides a topical composition which includes (a) about 1% by weight of pramoxine hydrochloride, (b) about 23.9% by volume of grape seed oil, (c) about 17.9% by volume of aloe vera, (d) about 12.8% by volume of lanolin, (e) about 8% by volume of shea butter, (f) about 3.6% by volume of emulsifying wax, (g) about 1.5% by volume of vitamin E, (h) about 0.75% by volume of grapefruit seed extract, (i) about 0.75% by weight of sodium borate (j) about 12 drops each of French lavender oil and Roman chamomile oil, and (k) water to make 100%.

The compositions of the present technology may be prepared by combining the water phase and oil phase components along with an emulsifier. Thus in one aspect the technology provides for method for preparing a topical composition, which includes heating the oil phase component and the water phase component, combining the two components to form an emulsion and cooling the emulsion. The oil phase component may include one or more natural butter, one or more carrier oil, one or more emollient and one or more emulsifier. The water phase component may include a pharmaceutically effective amount of a pramoxine compound, one or more moisturizers and water. The oil phase component and the water phase component may be heated for a suitable period of time at a suitable temperature to blend all the components. For example, the oil phase component and/or the water phase component may be heated to a suitable temperature, e.g., at about 200° C. or below, about 100° C. or below, about 80° C. or below, at about 75° C. or below, at about 70° C. or below, at about 65° C. or below, at about 60° C. or below, at about 55° C. or below, at about 50° C. or below, at about 45° C. or below, at about 40° C. or below or at about 30° C. or below, and ranges between and including any two of these values. In some embodiments, the oil phase component and/or the water phase component is heated to a temperature in the range of about 10° C. to about 150° C., about 20° C. to about 130° C., about 30° C. to about 110° C., about 40° C. to about 90° C., about 50° C. to about 70° C., about 45° C. to about 55° C. or about 55° C. to about 65° C., and ranges between and including any two of these values. In some embodiments, the oil phase component is heated to a temperature in the range of about 55° C. to about 65° C. In some embodiments, the water phase component is heated to a temperature in the range of about 45° C. to about 55° C. In some embodiments, the oil phase component is heated to about 70° C. or below. In some embodiments, the oil phase component is heated to about 60° C. In some embodiments, the water phase component is heated to about 50° C. Other additives may be added to either phases before, after or during heating.

The oil phase component and the water phase component may be combined by either adding the oil phase component to the water phase component or by adding the water phase component to the oil phase component, to obtain either an oil-in-water emulsion or a water-in-oil emulsion, respectively. In one embodiment, the water phase component is added to the oil phase component. In other embodiments, the oil phase component is added to the water phase component. The addition of one phase to another may be done rapidly or slowly over a period of time and may be accompanied by agitation or blending the combination using a suitable blending means. The final emulsion product may be cooled to a suitable temperature. Other additives may be added to the emulsion product before, after or during cooling.

In one embodiment, the method includes adding the oil phase components to a vessel and heating the vessel to about 55 to 75° C. with mixing. In some embodiments, the oil phase may be cooled to lower the temperature and various additives may be added. In some embodiments, water and other water phase components are combined and heated to about 45 to 55° C. and mixed in another vessel. In some embodiments, the water phase is then slowly added to the oil phase while mixing. In some embodiments, the blended emulsion mixture is cooled to about 35 to 45° C. In some embodiments, various functional additives and ingredients are added to the appropriate respective phases or the final emulsion. In some embodiments, the composition is shelf stable and can be stored at room temperature.

In some embodiments, the method further includes packaging the topical composition using suitable packaging methods known in the art using suitable packaging materials. As used herein, the term “packaging material” means any component of packaging in which the topical composition is contained. Packaging materials include, for example, polymer containers, tubes or glass containers, labels placed on or in packaging or on product, adhesives used to close or seal packaging or adhere labels and the like thereto; ink printed directly on product, directly on packaging, or on a label that is then adhered to packaging. In some embodiments, the packaging includes aseptic packaging. In some embodiments, the product is individually packed, (e.g., in individual tubes, plastic bags, bottles, sealed foils, etc.) and then collectively packed in bulk boxes. The packaged product can be stored or transported for commercial distribution and sale.

All of the ingredients or components used to prepare the compositions described herein are commercially available or can be synthesized using methods known in the art. The ingredients or components can be obtained in pure form or can be purified further to obtain the desired purity.

In one aspect, a method for topically treating painful skin conditions is provided, wherein the method includes applying the composition to skin wherein the composition includes a water phase component comprising a pharmaceutically effective amount of a pramoxine compound, a moisturizer and water; an oil phase component comprising a natural butter, a carrier oil and an emollient, and an emulsifier.

The present technology provides compositions and products to prevent and/or treat skin conditions that relieves pain and facilitates healing, while at the same time, protecting the skin against further breakdown by providing a superior protective barrier. Such skin conditions may include, for example, inflammatory skin conditions such as diaper rash, skin irritations, swelling, hemorrhoids, sunburn, windburn, minor burns, insect bites or stings, poison ivy, poison oak, poison sumac, skin allergies, minor cuts, scrapes or scratches, psoriasis, severe dryness, itchiness, stretch marks, acne, chapped or cracked skin, or microbial infection and disorders related to severe dry skin such as eczema. In one embodiment, the skin condition is diaper rash. Other embodiments include, but are not limited to supportive care in cases of simple sunburns, eczema, herpes zoster, poison plant dermatitis, as well as skin reactions following irritating cosmetic treatments such as laser therapy, chemical peels, and/or microdermabrasion.

As noted above, in one embodiment, the present compositions and methods provide a technique for adding a safe and effective anesthetic to a diaper cream. Diaper rash is an extremely common infant/child skin problem resulting in pain, irritability, doctor visits, sleepless nights, increased risk of secondary infections, as well as parental anxiety. Although diaper rash is an acute and superficial skin condition, it is often accompanied by pain, sensitivity, maceration and chaffing of the skin. When left untreated, the diaper rash can progress from inflammation to complicated infection caused by bacteria such as Staphylococcus aureus or yeasts such as Candida albicans. Until now, commercially available diaper creams typically provide only a protective skin barrier to repel moisture. These same creams have no component(s) to address the pain, nor provide components that facilitate healing.

When applied to the diaper area of a child with a diaper rash, the compositions of the present technology provide immediate relief from pain. By virtue of the additional components, the composition also serves to moisturize, nourish, hydrate, soothe, and heal the inflamed skin while also providing a barrier from further irritation by factors such as stool, urine, and diaper friction. The composition is safe, effective, and easy to apply and remove, and can be easily stored at room temperature. Although diaper rash is exemplified as one of the used, this technology maybe embodied in many different forms and should not be construed as limited to the embodiments set forth herein. The composition or formulation may be used, in one embodiment, for simple (contact) diaper dermatitis. Other embodiments could include, but are not limited to, adding to the composition described herein, a topical anti-fungal agent (such as nystatin) for yeast diaper dermatitis, or the addition of an anti-inflammatory (such as hydrocortisone) for more severe, highly inflamed diaper rashes.

The delivery of the composition is accomplished by formulating them in to a suitable form such as for example, lotion, cream, gel, ointment, or other solution or suspension, powder, concentrate, extract, medicated pads or wipes. The present compositions can be formulated into a wide variety of products. These include, but are not limited to, personal products such as diaper rash creams, skin creams, lotions, shaving creams, ointments, gels, antiseptics, soaps, shampoos, conditioners, insect repellant, etc. In some embodiments, the composition is a diaper rash cream. In some embodiments, the composition is a diaper rash lotion. In some other embodiments, the composition is a skin cream. In some embodiments, the composition is a skin lotion.

In some embodiments, the present compositions may include all naturally derived ingredients which are free of harsh chemicals. The present compositions may contain non-toxic and biodegradable ingredients. In some embodiments, the composition of the present technology is noncomedogenic, safe, natural, and leaves no oily film or residue. These compositions disclosed herein are, therefore, gentle enough to be used in all forms of compositions, e.g., as diaper rash cream.

Para. A. A topical composition comprising a water phase component comprising a pharmaceutically effective amount of a pramoxine compound, a moisturizer and water; an oil phase component comprising a natural butter, a carrier oil and an emollient; and an emulsifier.

Para. B. A topical composition comprising a water phase component comprising a pharmaceutically effective amount of a pramoxine compound, a moisturizer and water; and an oil phase component comprising a natural butter, a carrier oil, an emollient and an emulsifier.

Para. C. The topical composition of Para. A or B, wherein the pramoxine compound comprises pramoxine hydrochloride.

Para. D. The topical composition of Para. C, wherein the pramoxine hydrochloride is present in an amount of from about 0.25 to about 2 wt. % relative to the total volume of the composition.

Para. E. The topical composition of Para. C, wherein the pramoxine hydrochloride is present in an amount of from about 0.5 to about 1.5 wt. % relative to the total volume of the composition.

Para. F. The topical composition of Para. A or B, wherein the moisturizer is selected from the group consisting of aloe vera, glycerin, water soluble polyols, propylene glycol, polyethylene glycol, polypropylene glycol, sorbitol, and pantothenol, or a combination of two or more thereof.

Para. G. The topical composition of Para. F, wherein the moisturizer is aloe vera.

Para. H. The topical composition of Para. G, wherein the moisturizer is liquid aloe vera.

Para. I. The topical composition of Para. G, wherein the moisturizer is aloe barbadensis leaf juice.

Para. J. The topical composition of Para. I, wherein the moisturizer is pure aloe barbadensis leaf juice.

Para. J. The topical composition of Paras. F-J, wherein the moisturizer is present in an amount of from about 10 to about 25 volume % relative to the total volume of the composition.

Para. K. The topical composition of Para. A or B, wherein the natural butter is selected from the group consisting of shea butter, cocoa butter, aloe butter, olive butter, avocado butter, sweet almond butter, shealoe butter, coffee bean butter, cupuacu butter, coconut butter, hemp seed butter, kokum butter, macadamia nut butter, illipe butter, mango butter, mochacchino butter, murumuru butter, and pistachio nut butter, or a combination of two or more thereofthe moisturizer is present in an amount of from about 10 to about 25 volume % relative to the total volume of the composition

Para. L. The topical composition of Para. K, wherein the natural butter is shea butter.

Para. M. The topical composition of Paras. K-L, wherein the natural butter is present in an amount of from about 5 to about 10 volume % relative to the total volume of the composition.

Para. N. The topical composition of Para. A or B, wherein the emollient is selected from the group consisting of lanolin, lanolin derivatives mineral oil, petrolatum, castor oil, squalane, and silicons or a combination of two or more thereof.

Para. O. The topical composition of Para. N, wherein the emollient is lanolin.

Para. P. The topical composition of Paras. N-O, wherein the emollient is present in an amount of from about 5 to about 20 volume % relative to the total volume of the composition.

Para. Q. The topical composition of Para. A or B, wherein the emulsifier is selected from the group consisting of emulsifying wax, beeswax, polysorbates, ceteareths, glyceryl stearate, cetyl alcohol, cetearyl alcohol, cyclodextrins, cetyltrimethylammonium bromide, lecithin, polyoxyl castor oil, butylene glycol, and polyoxyethylene sorbitan, or a combination of two or more thereof.

Para. R. The topical composition of Para. Q, wherein the emulsifier is emulsifying wax.

Para. S. The topical composition of Paras. Q-R, wherein the emulsifier is present in an amount of from about 1 to about 10 volume % relative to the total volume of the composition.

Para. T. The topical composition of Para. A or B, wherein the one or more carrier oils are selected from the group consisting of grapeseed oil, sweet almond oil, olive oil, sunflower oil, rosehip seed oil, flax seed oil, safflower oil, argan oil, coconut oil, and avocado oil, or a combination of two or more thereof.

Para. U. The topical composition of Para. T, wherein the carrier oil is grapeseed oil.

Para. V. The topical composition of Paras. Q-R, wherein the carrier oil is present in an amount of from about 10 to about 40 volume % relative to the total volume of the composition.

Para. W. The topical composition of Para. A or B, further comprising one or more essential oils.

Para. X. The topical composition of Para. W, wherein the one or more essential oils are selected from the group consisting of lavender oil, chamomile oil, tea tree oil, clary sage oil, peppermint oil, spearmint oil and cinnamon oil.

Para. Y. The topical composition of Para. A or B, further comprising one or more components selected from a group consisting of antioxidants, preservatives, thickeners, fragrance agents and homogenizers.

Para. Z. The topical composition of Para. A or B, comprising pramoxine hydrochloride, shea butter, aloe vera, lanolin, grapeseed oil, emulsifying wax and water.

Para. AA. A composition comprising (a) about 0.25% to about 2% by weight of pramoxine hydrochloride, (b) about 10% to about 40% by volume of grapeseed oil, (c) about 10% to about 25% by volume of aloe vera, (d) about 5% to about 20% by volume of lanolin, (e) about 1% to about 20% by volume of shea butter,(f) about 1% to about 10% by volume of emulsifying wax, (g) about 0.1% to about 5% by volume of vitamin E, and (h) water to make 100%.

Para. BB. The topical composition of Para. AA, further comprising one or more preservatives and one or more essential oils.

Para. CC. The topical composition of Para. A or B, wherein the composition provides pain relief 4831-1790-0324.3

Para. DD. The topical composition of Para. A or B, wherein the composition is a diaper rash cream.

Para. EE. The composition of Para. AA, wherein the composition provides pain relief

Para. FF. The composition of Para. AA, wherein the composition is a diaper rash cream.

Para. GG. A method for topically treating painful skin conditions, the method comprising applying a topical composition to skin, wherein the composition comprises a water phase component comprising a pharmaceutically effective amount of a pramoxine compound, a moisturizer and water; an oil phase component comprising a natural butter, a carrier oil and an emollient and an emulsifier.

Para. HH. The method of Para. GG, wherein the painful skin condition comprises diaper rash.

Para. II. A method for preparing a topical composition, comprising heating a water phase component comprising a pharmaceutically effective amount of a pramoxine compound, a moisturizer and water; heating an oil phase component comprising a natural butter, a carrier oil, an emollient and an emulsifier; combining the water phase component and the oil phase component to form an emulsion; and cooling the emulsion.

Para. JJ. The method of Para. II, wherein the water phase is heated to a temperature of about 45° C. to about 55° C.

Para. KK. The method of Para. II, wherein the oil phase is heated to a temperature of about 55° C. to about 65° C.

Para. LL. The method of Para. II, wherein the water phase is heated to a temperature of about 50° C.

Para. MM. The method of Para. II, wherein the oil phase is heated to a temperature of about 60°

Para. NN. The method of Para. II, wherein the emulsion is cooled to a temperature of about 35 ° C. to about 45° C.

Para. OO. The method of Para. II, wherein the emulsion is cooled to a temperature of about 40° C.

Para. PP. The method of Para. II, wherein the oil phase component further comprises one or more preservatives and one or more antioxidants.

Para. QQ. The method of Para. II, wherein the water phase component further comprises one or more preservatives.

Para. RR. The method of Para. II, wherein one or more essential oils is added to the cooled emulsion.

As will be readily apparent to one of ordinary skill in the art, the concentration of a given ingredient can be increased or decreased beyond the range disclosed and the effect of the increased or decreased concentration can be determined using only routine experimentation. The optimal final concentrations for ingredients are typically identified by the nature of formulation and the desired end use of the composition.

The present technology, thus generally described, will be understood more readily by reference to the following examples which are provided by way of illustration and are not intended to be limiting in any way.

### EXAMPLES

The present technology is further illustrated by the following examples which should not be construed as limiting in any way.

Illustrative compositions of present technology, for use as a diaper rash cream, are described in Example 1. It is understood that larger batches of each of the compositions can be made simply by increasing the amount of each component by the same factor. For example, a double-batch can be made by increasing the amount of each component by 2; a triple batch can be made by increasing the amount of each component by three, a ten-fold batch can be made by increasing the amount of each component by 10, etc. The ratio of components remains essentially constant while the volume of each increases. The compositions can be formulated as concentrates. In some embodiments, the concentrates include no water or water up to 95%, 96%, 97%, 98% or 99% of the amount provided in the formulations described below. Additionally or alternatively, in some embodiments, the individual components are combined and water is added such that the final volume is 100%. Except for the water, in such embodiments, the ratio of the components will remain essentially the same.

### Example 1

### Cream Composition (A)

The present composition was prepared with an oil phase and water phase component, which when blended together with an emulsifier, produce a cream that can be easily applied to the skin.

### Oil Phase Component

The oil phase component was produced by dissolving 43 mL of purified lanolin, 27 mL of hydro-dispersible shea butter and 12 g of emulsifying wax NF in 80 mL of natural grape seed oil in a 600 mL beaker and heated to 60° C. on a hot plate. The temperature of the hot plate and that of the mixture was then reduced to 50° C. and 5 mL of vitamin E oil and 2.5 mL of grapefruit seed extract was added to the mixture and stirred.

### Water Phase Component

The water phase component was prepared by mixing 100 mL of purified water with 60 mL of the 100% pure aloe vera liquid in a 250 mL flask and heating it on a hot plate to 50 ° C. followed by addition of 3.5 g of pramoxine and 2.5 g of sodium borate and stirring until completely dissolved.

The two components were combined together by slowly pouring the water phase mixture into the oil phase mixture while blending the combination with the hand-held blender (Cuisinart Smart Stick®) for 60 seconds. The mixture was then cooled to 40° C. and 12 drops of French lavender essential oils and 12 drops of Roman chamomile essential oils were added and the mixture was again stirred. The final product was then divided into 50 mL aliquots and poured into opaque plastic jars which were covered with air-tight lids and allowed stand for 24 hours.

### Example 2

### Cream Composition (B)

The present composition was prepared with an oil phase and water phase component, which when blended together with an emulsifier, produce a cream that can be easily applied to the skin.

### Oil Phase Component

The oil phase component was produced by 40 mL of 100% naturally refined hydro-dispersible shea butter in a 600 ml beaker heated on a hot plate to 70° C. To this heated liquid, 20 mL of wax (100% pure white beeswax +/−emulsifying wax NF) was added and stirred until dissolved. 80 mL of a combination of 100% natural jojoba and 100% natural grape seed oil was added to the mixture and stirred, as was 30 mL of 100% lanolin. The mixture was then removed from the heat and allowed to cool to 50° C., and 5 mL of 100% vitamin E oil and 2.5 mL of grapefruit seed extract (60%} was stirred in.

### Water Phase Component

The water phase component was prepared by heating 100 mL of purified water mixed with 60 mL of 100% pure aloe vera liquid to 50° C. in a 250 mL flask on a hot plate in which 3.5 grams of pramoxine and 2.5 grams of sodium borate were added and stirred until completely dissolved.

The two components were then combined together by slowly pouring the water-based mixture into the oil-based mixture while blending the combination with a hand-held blender (Cuisinart Smart Stick®} over a 60 second period. The mixture was then left to cool to 40° C., and 10 drops of French lavender essential oil and 10 drops of Roman chamomile essential oil were added and stirred. The final product was divided into 100 mL aliquots and poured into 4 oz. opaque glass or plastic jars which were covered with lids and let stand for 24 hours.

The final product can then be applied to the affected skin after cleansing and drying, once or several times a day, until the skin condition is resolved.

While certain embodiments have been illustrated and described, it should be understood that changes and modifications can be made therein in accordance with ordinary skill in the art without departing from the technology in its broader aspects as defined in the following claims.

The embodiments, illustratively described herein, may suitably be practiced in the absence of any element or elements, limitation or limitations, not specifically disclosed herein. Thus, for example, the terms “comprising,” “including,” “containing,” etc., shall be read expansively and without limitation. Additionally, the terms and expressions employed herein have been used as terms of description and not of limitation, and there is no intention in the use of such terms and expressions of excluding any equivalents of the features shown and described, or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art, all language such as “up to,” “at least,” “greater than,” “less than,” and the like, includes the number recited and refers to ranges which can be subsequently broken down into subranges as discussed above. Finally, as will be understood by one skilled in the art, a range includes each individual member.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A topical composition comprising:
a water phase component comprising a pharmaceutically effective amount of a pramoxine compound, a moisturizer and water;
an oil phase component comprising a natural butter, a carrier oil and an emollient; and
an emulsifier.

2. The composition of claim 1, wherein the pramoxine compound comprises pramoxine hydrochloride.

3. The composition of claim 2, wherein the pramoxine hydrochloride is present in an amount of from about 0.25 to about 2 wt. % relative to the total volume of the composition.

4. The composition of claim 1, wherein the moisturizer is selected from the group consisting of aloe vera, glycerin, water soluble polyols, propylene glycol, polyethylene glycol, polypropylene glycol, sorbitol, and pantothenol, or a combination of two or more thereof.

5. The composition of claim 4, wherein the moisturizer is aloe vera and is present in an amount of from about 10 to about 25 volume % relative to the total volume of the composition.

6. The composition of claim 1, wherein the natural butter is selected from the group consisting of shea butter, cocoa butter, aloe butter, olive butter, avocado butter, sweet almond butter, shealoe butter, coffee bean butter, cupuacu butter, coconut butter, hemp seed butter, kokum butter, macadamia nut butter, illipe butter, mango butter, mochacchino butter, murumuru butter, and pistachio nut butter, or a combination of two or more thereof.

7. The composition of claim 6, wherein the natural butter is shea butter and is present in an amount of from about 5 to about 10 volume % relative to the total volume of the composition.

8. The composition of claim 1, wherein the emollient is selected from the group consisting of lanolin, lanolin derivatives mineral oil, petrolatum, castor oil, squalane, and silicons or a combination of two or more thereof.

9. The composition of claim 8, wherein the emollient is lanolin and is present in an amount of from about 5 to about 20 volume % relative to the total volume of the composition.

10. The composition of claim 1, wherein the emulsifier is selected from the group consisting of emulsifying wax, beeswax, polysorbates, ceteareths, glyceryl stearate, cetyl alcohol, cetearyl alcohol, cyclodextrins, cetyltrimethylammonium bromide, lecithin, polyoxyl castor oil, butylene glycol, and polyoxyethylene sorbitan, or a combination of two or more thereof.

11. The composition of claim 10, wherein the emulsifier is emulsifying wax and is present in an amount of from about 1 to about 10 volume % relative to the total volume of the composition.

12. The composition of claim 1, wherein the one or more carrier oils are selected from the group consisting of grape seed oil, sweet almond oil, olive oil, sunflower oil, rosehip seed oil, flax seed oil, safflower oil, argan oil, coconut oil, and avocado oil, or a combination of two or more thereof.

13. The composition of claim 12 wherein the carrier oil is grape seed oil and is present in an amount of from about 10 to about 40 volume % relative to the total volume of the composition.

14. The composition of claim 1, further comprising one or more essential oils selected from the group consisting of lavender oil, chamomile oil, tea tree oil, clary sage oil, peppermint oil, spearmint oil and cinnamon oil.

15. The composition of claim 1, comprising one or more components selected from a group consisting of antioxidants, preservatives, thickeners, fragrance agents and homogenizers.

16. The composition of claim 1, wherein the composition comprises pramoxine hydrochloride, shea butter, aloe vera, lanolin, grape seed oil, emulsifying wax and water.

17. The composition of claim 16 comprising:
(a) about 0.25% to about 2% by weight of pramoxine hydrochloride,
(b) about 10% to about 40% by volume of grape seed oil,
(c) about 10% to about 25% by volume of aloe vera,
(d) about 5% to about 20% by volume of lanolin,
(e) about 1% to about 20% by volume of shea butter,
(f) about 1% to about 10% by volume of emulsifying wax,
(g) about 0.1% to about 5% by volume of vitamin E, and
(h) water to make 100%.

18. The composition of claim 17, further comprising one or more preservatives and one or more essential oils.

19. A method for topically treating painful skin conditions, the method comprising applying a topical composition to skin,
wherein the composition comprises
a water phase component comprising a pharmaceutically effective amount of a pramoxine compound, a moisturizer and water;
an oil phase component comprising a natural butter, a carrier oil and an emollient; and
an emulsifier.

20. A method for preparing a topical composition, comprising:
heating a water phase component comprising a pharmaceutically effective amount of a pramoxine compound, a moisturizer and water;
heating an oil phase component comprising a natural butter, a carrier oil, an emollient and an emulsifier;
combining the water phase component and the oil phase component to form an emulsion; and
cooling the emulsion.
